(19) United States
(12) Patent Application Publication
(10) Pub. No.: US 2023/0059710 A1
(43) Pub. Date: Feb. 23, 2023

(54) SURGICAL STAPLING INSTRUMENT WITH CURVED END EFFECTOR ASSEMBLY

(71) Applicant: Covidien LP, Mansfield, MA (US)

(72) Inventors: Syed Sarfraz Ahamed, Shanghai (CN); Xini Zhang, Shanghai (CN)

(21) Appl. No.: 17/796,922

(22) PCT Filed: Feb. 3, 2020
(86) PCT No.: PCT/CN2020/074185

§ 371 Date: Aug. 2, 2022

(51) Int. Cl. A61B 17/072

(52) U.S. Cl. CPC A61B 17/07207; A61B 2017/07271; A61B 2017/07264; A61B 2017/0688

(57) ABSTRACT

A curved cartridge assembly of a surgical stapler includes a staple cartridge, an alignment pin received in the staple cartridge and configured to engage an opening in an anvil assembly, and a shipping cap configured to detachably couple to the staple cartridge. The shipping cap has is engaged with the distal end portion of the alignment pin to resist translation of the alignment pin. With the cartridge assembly loaded into the surgical stapler, upon releasing the shipping cap from the staple cartridge the alignment pin translates into engagement with the opening in the anvil assembly.

### CROSS-REFERENCE TO RELATED APPLICATION

This application is a National Stage Application of PCT Application No. PCT/CN2020/074185 under 35 USC § 371(a), filed Feb. 3, 2020, the entire contents of which are incorporated by reference herein.

### FIELD

The disclosure relates generally to a surgical instrument and, more specifically, to a surgical stapling instrument for clamping, joining, and/or cutting tissue.

### BACKGROUND

Certain surgical stapling instruments are used for applying rows of staples through compressed living tissue. These surgical stapling instruments are employed, for example, for fastening tissue or organs prior to transection or resection or during anastomoses. In some cases, these surgical stapling instruments are utilized for occluding organs in thoracic and abdominal procedures.

Typically, such surgical stapling instruments include an anvil assembly, a cartridge assembly for supporting an array of surgical staples, an approximation mechanism for approximating the cartridge and anvil assemblies, an alignment or guide pin assembly for capturing tissue between the cartridge and anvil assemblies and/or for maintaining alignment between the cartridge and anvil assemblies during approximation and firing, and a firing mechanism for ejecting the surgical staples from the cartridge assembly.

In use, a surgeon initially advances the alignment pin assembly and subsequently approximates the anvil and cartridge assemblies to clamp tissue between the cartridge and anvil assemblies. In some surgical stapling instruments, the alignment pin is automatically advanced in response to approximation of the anvil and cartridge assemblies. Next, the surgeon fires the surgical stapling instrument to place staples in tissue. Optionally, the surgeon may use the same surgical stapling instrument or a separate device to cut tissue positioned adjacent or between the rows of staples. In some instances, the surgical stapling instrument includes a knife which cuts tissue as the staples are fired.

### SUMMARY

In one aspect of the disclosure, a curved cartridge assembly is provided and includes a staple cartridge having a tissue-contacting surface, an alignment pin, and a shipping cap. The staple cartridge defines a plurality of rows of staple slots and a channel extending through the tissue-contacting surface. The alignment pin is received in the channel and has a distal end portion that protrudes distally from the tissue-contacting surface of the staple cartridge. The alignment pin is movable within the channel from a retracted position to an advanced position. The shipping cap is detachably coupled to the staple cartridge to cover the tissue-contacting surface of the staple cartridge. The shipping cap has an end portion defining a notch that receives the distal end portion of the alignment pin. The shipping cap engages the alignment pin to resist translation of the alignment pin along a longitudinal axis from the retracted position towards the advanced position.

In aspects, the distal end portion of the alignment pin may define a groove, and the end portion of the shipping cap may have a tab extending into the notch. The tab may be received within the groove of the alignment pin to resist translation of the alignment pin from the retracted position towards the advanced position.

In aspects, the groove of the alignment pin may extend at least partially around a circumference of the alignment pin.

In aspects, the staple cartridge may further include a leg extending proximally from an end portion thereof, and the shipping cap may further include a plate and a leg extending proximally from the plate. The plate of the shipping cap may be configured to cover the tissue-contacting surface, and the leg of the shipping cap may be configured to detachably secure to the leg of the staple cartridge.

In aspects, the shipping cap may be configured to detach from the staple cartridge in a direction from a first end portion of the staple cartridge toward a second end portion of the staple cartridge.

In aspects, the shipping cap may be configured to slide along the tissue-contacting surface of the staple cartridge as the shipping cap is detached from the staple cartridge to disengage from the distal end portion of the alignment pin.

In accordance with another aspect of the disclosure, an end effector assembly of a surgical stapler is provided and includes a curved housing having a base portion and a jaw portion, an anvil assembly supported on the jaw portion, a curved staple cartridge configured for insertion into the base portion, an alignment pin supported in the curved staple cartridge, and a shipping cap detachably coupled to the staple cartridge. The anvil assembly has a tissue-contacting surface defining a plurality of staple-forming pockets and an opening, and the curved staple cartridge includes a tissue-contacting surface defining a plurality of staple slots. The alignment pin includes a distal end portion that protrudes distally from the tissue-contacting surface of the curved staple cartridge. The distal end portion of the alignment pin is configured for receipt in the opening of the anvil assembly. The shipping cap is engaged with the distal end portion of the alignment pin to resist axial movement of the alignment pin relative to the curved staple cartridge.

In aspects, the distal end portion of the alignment pin may define a groove, and the shipping cap may have a tab removably received in the groove.

In aspects, the shipping cap may be configured to slide along a plane defined by the tissue-contacting surface of the staple cartridge to disengage from the distal end portion of the alignment pin.

In aspects, the end effector assembly may further include a slider slidably received in the curved housing. The slider may have a camming surface configured to engage a proximal end portion of the alignment pin to resiliently bias the distal end portion of the alignment pin toward the opening in the anvil assembly.

In aspects, the proximal end portion of the alignment pin may be configured to cam the slider proximally from a distal position to a proximal position during insertion of the staple cartridge into the curved housing.

In aspects, the alignment pin may be configured to translate distally to position the distal end portion of the alignment pin in the opening of the anvil assembly in response to the slider moving distally from the proximal position to the distal position.

In aspects, the slider may be configured to move from the proximal position to the distal position in response to the shipping cap being detached from the curved staple cartridge.

In aspects, the end effector assembly may further include a shaft disposed in the curved housing and a spring supported on the shaft. The slider may be slidably supported on the shaft, and the spring may be engaged with the slider to resiliently bias the slider into engagement with the alignment pin.

In aspects, the end effector assembly may further include a bushing fixed about a proximal end portion of the alignment pin, and a thrust bar disposed in the curved housing and configured to move between a proximal position and a distal position. The thrust bar may have a distal end portion configured to engage the bushing.

In aspects, the bushing and the alignment pin may be configured to translate distally to position the distal end portion of the alignment pin in the opening of the anvil assembly in response to the thrust bar moving from the proximal position to the distal position.

In aspects, the bushing may have a chamfered surface configured to engage the thrust bar during insertion of the curved staple cartridge into the curved housing.

In aspects, the thrust bar may be configured to unfix the bushing from the alignment pin to move the bushing distally along the alignment pin upon further distal movement of the thrust bar from the distal position.

In aspects, the distal end portion of the thrust bar may define a channel configured for receipt of the proximal end portion of the alignment pin as the thrust bar moves distally from the distal position.

In aspects, the bushing and the alignment pin may move distally as the thrust bar moves distally from the proximal position to the distal position, and the bushing and the thrust bar may move distally relative to the alignment pin as the thrust bar moves distally from the distal position.

### BRIEF DESCRIPTION OF DRAWINGS

Various aspects of the disclosed surgical stapling instrument are disclosed herein with reference to the drawings, wherein:

FIG. 1 is a side, perspective view illustrating an exemplary surgical stapler having a curved end effector assembly;

FIG. 2 is a perspective view, with parts disassembled, illustrating a staple cartridge assembly and a curved housing of the end effector assembly of FIG. 1;

FIG. 3 is a perspective view illustrating the staple cartridge assembly of FIG. 2 including a staple cartridge and a shipping cap;

FIG. 4 is an enlarged view of the area of detail labeled “4” in FIG. 3 illustrating a connection between the shipping cap and an alignment pin of the staple cartridge;

FIG. 5 is a perspective view illustrating the shipping cap of FIG. 3;

FIG. 6 is an enlarged view of the area of detail labeled “6” in FIG. 5 illustrating a notch of the shipping cap;

FIG. 7 is a cross-sectional view, taken along line 7-7 in FIG. 3, illustrating the alignment pin engaged with the shipping cap;

FIG. 8 is a perspective view illustrating the staple cartridge assembly in a first insertion state with the curved housing of the surgical stapler;

FIG. 9 is a perspective view illustrating the staple cartridge assembly in a second insertion state with the curved housing;

FIG. 10 is a cross-sectional view, taken along line 10-10 in FIG. 9, illustrating the alignment pin engaged between the shipping cap and a pin alignment mechanism;

FIG. 11 is a perspective view of the pin alignment mechanism of FIG. 10;

FIG. 12 is an enlarged view of the area of detail labeled “12” in FIG. 1 illustrating the alignment pin engaged with the anvil assembly after having detached the shipping cap from the staple cartridge;

FIG. 13 is a cross-sectional view, taken along line 13-13 in FIG. 12, illustrating the alignment pin held in engagement with the anvil assembly by the pin alignment mechanism;

FIG. 14 is a perspective view, with parts disassembled, illustrating an end effector assembly having an alternate alignment pin;

FIG. 15 is a perspective view of the alignment pin of FIG. 14;

FIG. 16 is a cross-sectional view, taken along line 16-16 in FIG. 14, illustrating the alignment pin engaged with the shipping cap;

FIG. 17 is a perspective view illustrating the end effector assembly of FIG. 14 in an assembled state after the shipping cap has been removed;

FIG. 18 is a cross-sectional view, taken along line 18-18 in FIG. 17, illustrating a thrust bar of the end effector assembly in a proximal position;

FIG. 19 is a perspective view illustrating the thrust bar of FIG. 18 engaged with a bushing of the alignment pin; and

FIG. 20 is longitudinal cross-sectional view of the end effector assembly of FIG. 14 illustrating the thrust bar in a distal position, in which the alignment pin is engaged with the anvil assembly.

### DETAILED DESCRIPTION

Aspects of the disclosed surgical stapling instrument are described in detail with reference to the drawings, wherein like reference numerals designate corresponding elements in each of the several views. In the drawings and the description that follows, the term “proximal” refers to the end of the surgical stapling instrument that is closer to the clinician, whereas the term “distal” refers to the end of the surgical stapling instrument that is farther from the clinician. In addition, the term “clinician” is used generally to refer to medical personnel including doctors, nurses, and support personnel.

It should be appreciated that the instrument described and illustrated herein is configured to fire surgical staples against an anvil surface; however, aspects of the disclosure are equally applicable with other forms of staples, fasteners, clips, as well as two part fasteners, made of metallic or polymeric material.

With surgical staplers, it is important that the staple slots of the staple cartridge are aligned with the staple-forming pockets of the anvil assembly so that staple formation occurs properly. Incorrect alignment may lead to staple malformation and/or tissue trauma. An alignment pin is provided that assists in aligning the staple slots of the staple cartridge with the staple-forming pockets of the anvil assembly and also acts as a stop for preventing excess tissue from entering a gap between the cartridge assembly and the anvil assembly to ensure that staple formation and tissue cutting occur in close proximity to one another. A shipping cap is also provided that is detachably coupled to the staple cartridge of the cartridge assembly to cover the staple slots and also hold the alignment pin in an axial position until the staple cartridge is fully loaded into the surgical stapler. Upon loading the staple cartridge into the surgical stapler, the shipping cap may be detached from the staple cartridge, whereby the alignment pin can be translated into engagement with the anvil assembly to maintain proper alignment of the staple cartridge and the anvil assembly with one another.

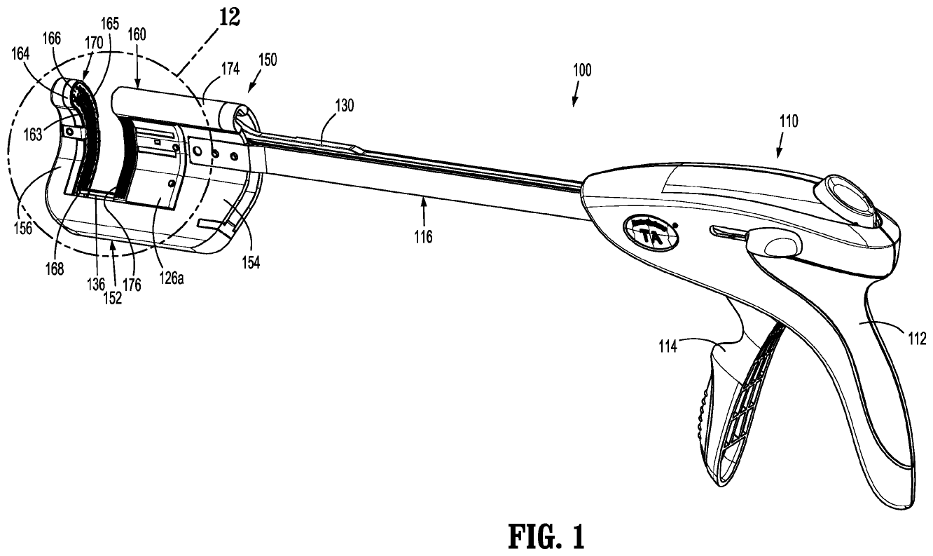

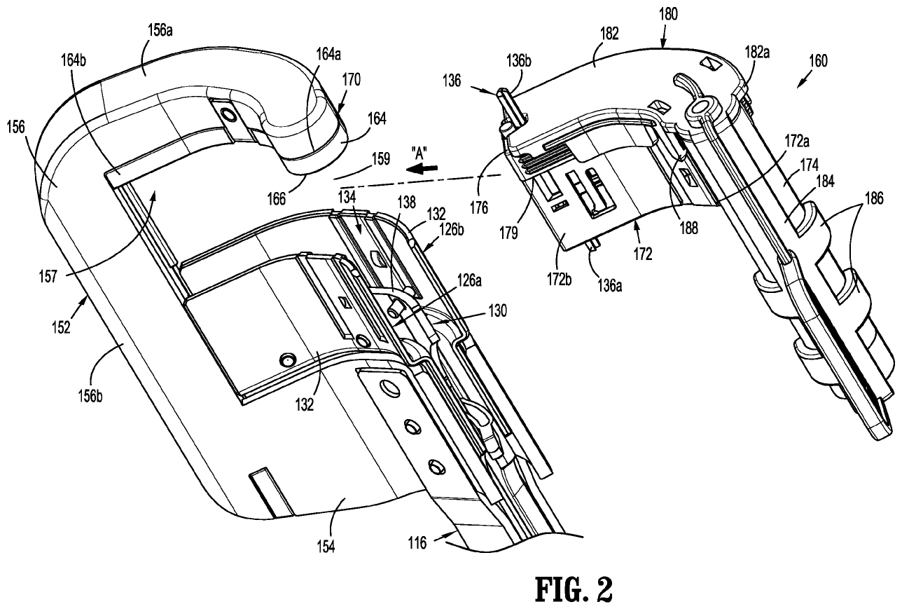

With reference FIGS. 1 and 2, a surgical stapling instrument according to an aspect of the disclosure is shown generally as surgical stapling instrument 100. The surgical stapling instrument 100 includes a handle assembly 110 having a stationary handle or base 112, a trigger member 114 pivotally secured relative to the stationary handle 112, and an elongated body portion 116 extending distally from the stationary handle 112. An end effector assembly 150 is secured to a distal portion of the elongated body portion 116 and includes a staple cartridge assembly 160 and an anvil assembly 170. In response to an actuation of the trigger member 114, the cartridge assembly 160 is configured to move between an open position, in which the cartridge assembly 160 is spaced from the anvil assembly 170, and a fully approximated or clamped position, in which the cartridge assembly 160 is disposed adjacent to the anvil assembly 170.

A pair of clamp slide members 126a, 126b, and a thrust bar 130 are slidably supported in the elongated body portion 116 for movement between retracted and advanced positions in response to movement of the trigger member 114 through an approximation stroke and/or a firing stroke. A distal portion of each clamp slide member 126a, 126b includes a head portion 132 together defining a cavity 134 configured to releasably receive and support the cartridge assembly 160 of the end effector assembly 150.

For a detailed description of various aspects of the handle assembly 110 and the mechanical linkages responsible for carrying out a stapling function of the end effector assembly 150, please refer to commonly owned U.S. Patent Application Publication No, 2018/0153544, filed on Nov. 30, 2017, the contents of which are hereby incorporated by reference herein in its entirety.

The end effector assembly 150 of the surgical stapling instrument 100 includes a housing 152 having a base portion 154 coupled to and extending from a distal end portion of the elongated body portion 116 and an L-shaped jaw portion 156 extending distally from the base portion 154. The housing 152 of the end effector assembly 150 includes a substantially curved, C-like, or hook shaped cross-section. In aspects, the housing 152 may be linear rather than curved. The L-shaped jaw portion 156 includes an anvil support 156a that is spaced from and overlaps with the base portion 154, and an elongated arm 156b extending between and interconnecting the anvil support 156a and the base portion 154. The head portion 132 of each of the clamp slide members 126a, 126b and a head portion 138 of the thrust bar 130 extend through the housing 152 and are received in a gap 157 defined between the base portion 154 and the anvil support 156a of the L-shaped jaw portion 156.

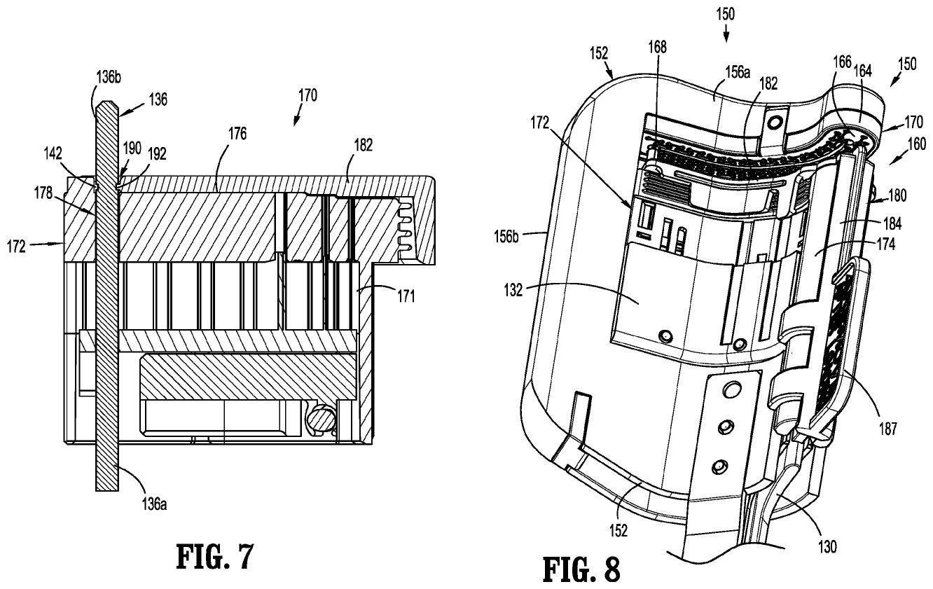

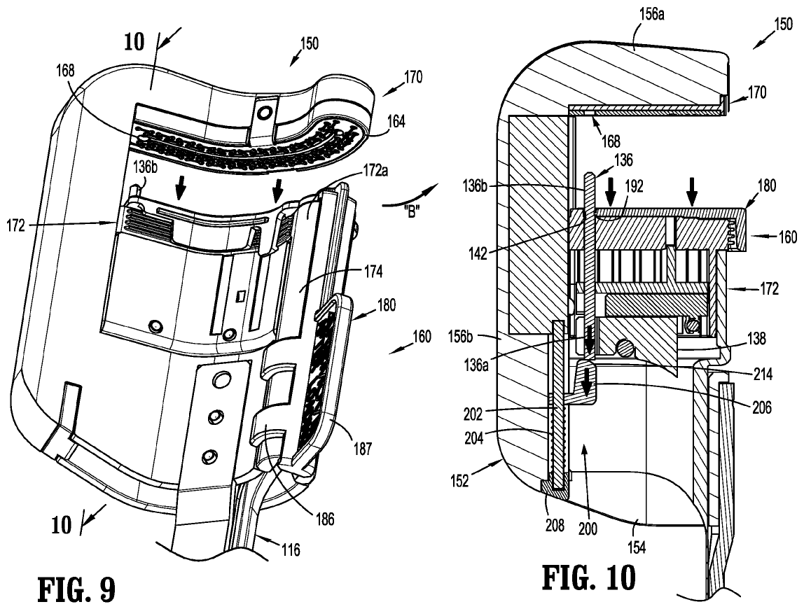

The anvil assembly 170 of the end effector assembly 150 includes an anvil member 164 supported on the anvil support 156a of the L-shaped jaw portion 156 of the housing 152 and defines the same curved profile as the anvil support 156a. The anvil member 164 has a front end portion 164a disposed adjacent a lateral opening 159 of the gap 157, and a rear end portion 164b disposed adjacent the elongated arm 156h of the L-shaped jaw portion 156. The anvil member 164 has a tissue-contacting surface 166 extending between the front and rear end portions 164a, 164b of the anvil member 164. The tissue-contacting surface 166 defines a longitudinal slot 163 (FIG. 1) configured for receipt of a knife member (not explicitly shown) of the cartridge assembly 160, and an opening 168 (see also FIG. 9) at the rear end portion 164b of the anvil member 164. The opening 168 is configured to receive a distal end portion 136b of a bottom or rear end alignment pin 136. The tissue-contacting surface 166 of the anvil member 164 further defines first and second sets of staple pockets 165. The position of the first and second sets of staple-forming pockets 165 correspond to a position of first and second arrays of staple receiving slots 171 (FIG. 7) of a staple cartridge 172 of the cartridge assembly 160.

The cartridge assembly 160 of the end effector assembly 150 of the surgical stapling instrument 100 (FIG. 1) is releasably supported within the cavity 134 between the clamp slide members 126a, 126b and includes the staple cartridge 172 and a shipping cap 180 detachably coupled to the staple cartridge 172. The staple cartridge 172 has the same curved profile as the base portion 154 of the housing 152. The staple cartridge 172 has a front end portion 172a, a rear end portion 172b, a leg 174 extending proximally from the front end portion 172a, and a tissue-contacting surface 176. The tissue-contacting surface 176 is configured to face the tissue-contacting surface 166 of the anvil member 164 when the staple cartridge 172 is assembled to the base portion 154 of the housing 152. The rear end portion 172h of the staple cartridge 172 defines a channel 178 (FIG. 7) that extends through the tissue-contacting surface 176 in a direction parallel with a longitudinal axis of the elongated body portion 116.

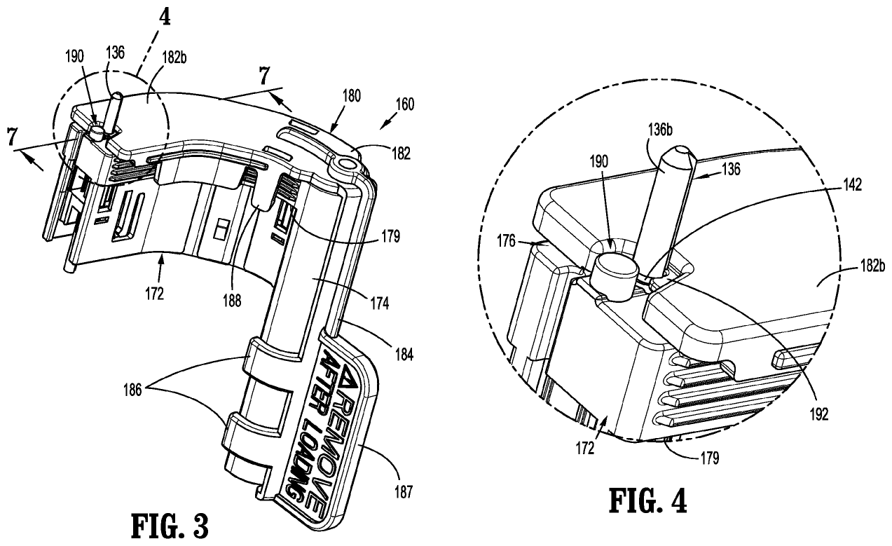

With reference to FIGS. 2-4, the alignment pin 136 is received through the channel 178 (FIG. 7) of the staple cartridge 172 and has a proximal end portion 136a and the distal end portion 136b. The distal end portion 136b of the alignment pin 136 protrudes distally from the tissue-contacting surface 176 of the staple cartridge 172 and is configured for receipt in the opening 168 (FIGS. 8 and 9) of the anvil member 164 upon the staple cartridge 172 being fully assembled to the housing 152 and after the alignment pin 136 moves to an advanced position. The distal end portion 136b of the alignment pin 136 defines an annular groove 142 (FIG. 4) that extends around the circumference of the alignment pin 136.

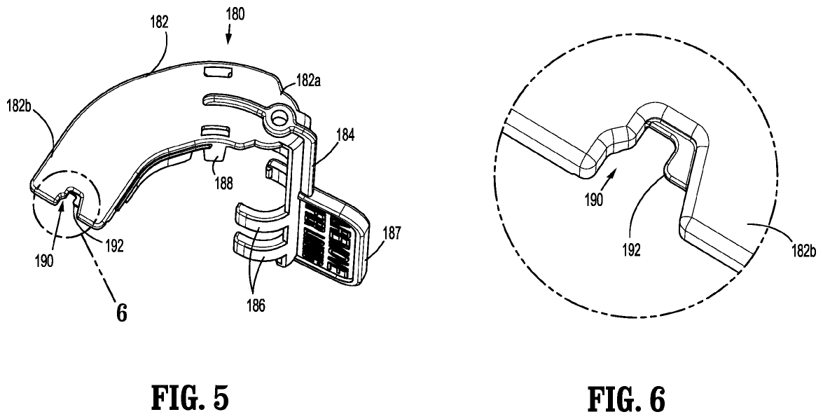

With reference to FIGS. 2-7, the shipping cap 180 of the staple cartridge assembly 160 is detachably coupled to the staple cartridge 172 and generally includes a cover plate 182 and a leg 184 extending proximally from a front end portion 182a of the cover plate 182. The leg 184 includes one or more securement fingers 186 each having a curved shape and being configured to flex outwardly to receive the leg 174 of the staple cartridge 172 in a snap-fit engagement. The leg 184 has a tab 187 extending laterally outward therefrom that is configured to be grasped between two fingers of a clinician to facilitate detachment and removal of the shipping cap 180 from the staple cartridge 172. The cover plate 182 of the shipping cap 180 is positioned over the tissue-contacting surface 176 of the staple cartridge 172 to cover the staple-forming pockets 171 (FIG. 7). The cover plate 182 may have a pair of hooks 188 extending proximally from opposite sides thereof for detachably engaging a rib 179 of respective side walls of the staple cartridge 172.

The cover plate 182 of the shipping cap 180 has a rear end portion 182b defining a notch 190 for receiving the distal end portion 136b of the alignment pin 136. The rear end portion 182b of the cover plate 182 has a tab 192, such as, for example, a catch, that extends into the notch 190 and is received in the groove 142 of the alignment pin 136. The tab 192 of the shipping cap 180, when engaged with the alignment pin 136, maintains the alignment pin 136 in a fixed axial position relative to the staple cartridge 172 out of engagement with the anvil assembly 170, as shown in FIG. 8. In other aspects, the shipping cap 180 may have a recess and the alignment pin 136 may have a tab or projection received in the recess of the shipping cap 180 for selectively fixing the axial position of the alignment pin 136.

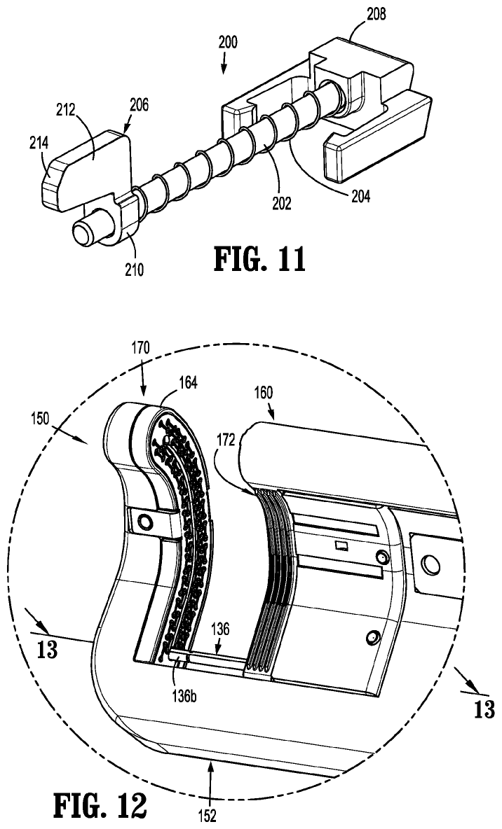

With reference to FIGS. 10, 11, and 13, the end effector assembly 150 further includes a pin alignment mechanism 200 disposed within the base portion 154 of the housing 152 of the end effector assembly 150. The pin alignment mechanism 200 includes a shaft 202 axially fixed in the base portion 154 of the housing 152, a biasing member 204 (e.g., a spring) supported on the shaft 202, a slider 206 slidably supported on the shaft 202, and a fixture 208 fixedly secured to the base portion 154 of the housing 152. The biasing member 204 is compressed between the slider 206 and the fixture 208 of the pin alignment mechanism 200 to resiliently bias the slider 206 in a distal direction along the shaft 202. The slider 206 has a shroud 210 (FIG. 11) through which the shaft 204 extends and a flange 212 extending from the shroud 210. The flange 212 of the slider 206 has a distally-facing camming surface 214 that is engaged with the proximal end portion 136a of the alignment pin 136 when the staple cartridge 172 is received in the housing 152 of the end effector 150 to exert a distally-oriented resilient bias, via the biasing member 204, on the alignment pin 136 of the cartridge assembly 160.

In assembly, with initial reference to FIG. 2, the staple cartridge 172 and the attached shipping cap 180 are inserted into the gap 157 of the housing 152 of the end effector 150 via the opening 159 and the cavity 134 defined between the clamp slide members 126a, 126b, in a direction indicated by arrow “A,” to position the staple cartridge 172 in spaced, opposing relation with the anvil assembly 170 (FIG. 8). To fully insert the staple cartridge 172 in the housing 152, the cartridge assembly 160 is translated proximally (e.g., away from the anvil assembly 170) from the position shown in FIG. 8 to the position shown in FIG. 10. Since the tab 192 of the shipping cap 180 is disposed in the groove 142 of the alignment pin 136, axial movement of the alignment pin 136 is resisted during loading of the cartridge assembly 160 into the housing 152.

While moving the cartridge assembly 160 (the staple cartridge 172 and the shipping cap 180) proximally to the position shown in FIG. 10, the proximal end portion 136a of the alignment pin 136 engages the camming surface 214 of the slider 206 of the pin alignment mechanism 200 to move the slider 206 against the resilient bias of the biasing member 204 along the shaft 202 from a starting or distal position toward a proximal position.

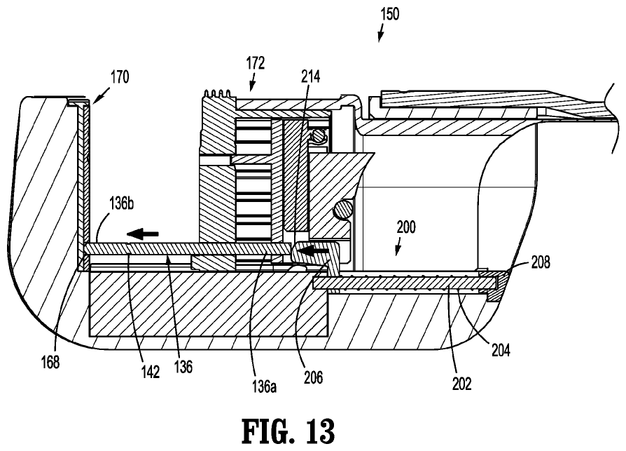

To complete the assembly process, the projection 187 of the shipping cap 180 may be grasped and pulled by a clinician with sufficient force to disengage the fingers 186 of the shipping cap 180 from the leg 174 of the staple cartridge 172. The shipping cap 180 may then be slid along the staple cartridge 172 toward the front end portion 172a of the staple cartridge in the direction indicated by arrow “B” in FIG. 9. Upon removing the shipping cap 180 from the staple cartridge 172, the tab 192 (FIG. 10) of the shipping cap 180 is removed from within the groove 142 in the alignment pin 136 and no longer prohibits axial movement of the alignment pin 136 within the staple cartridge 172. With the alignment pin 136 freed from the shipping cap 180, the distally-oriented force of the biasing member 204 of the pin alignment mechanism 200 exerted on the alignment pin 136 via the slider 206 moves the alignment pin 136 along the shaft 202 from the proximal position, as shown in FIG. 10, to the distal position, as shown in FIGS. 12 and 13. In the distal position, the distal end portion 136b of the alignment pin 136 is received in the opening 168 of the anvil member 164 to maintain the staple cartridge 172 and the anvil member 164 in proper longitudinal alignment with one another.

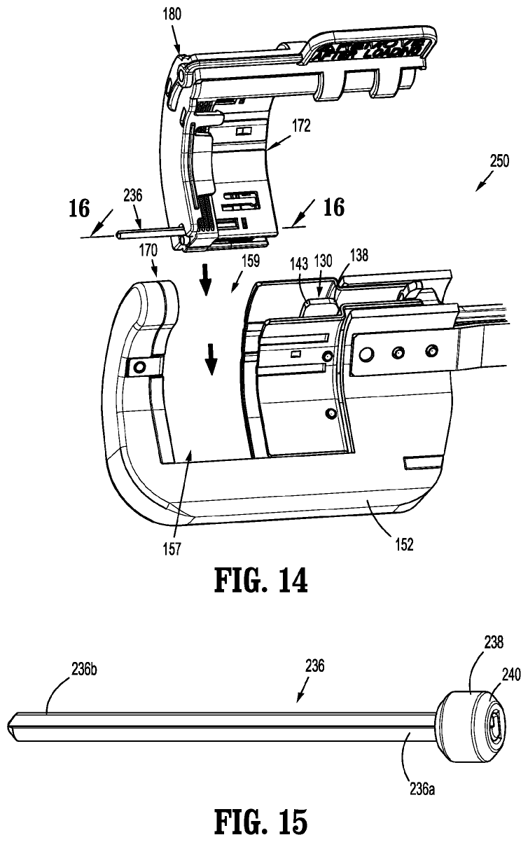

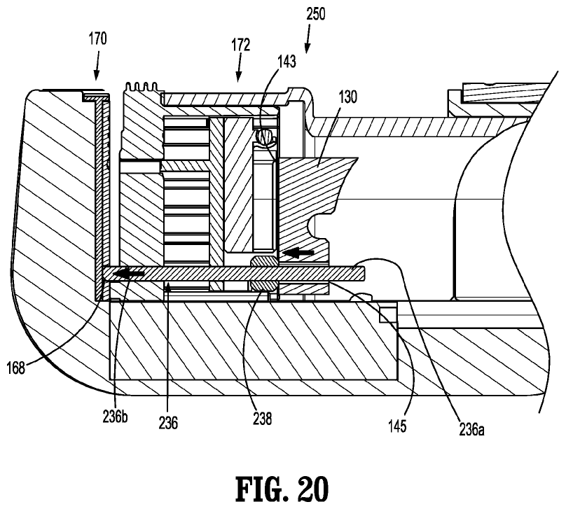

FIGS. 14-20 illustrate another end effector assembly 250, similar to the end effector assembly 150 of FIGS. 1-13. The end effector assembly 250 differs from the end effector assembly 150 by having an alternate version of the alignment pin 136 shown generally as alignment pin 236.

The alignment pin 236 of the end effector assembly 250 includes a distal end portion 236b, a proximal end portion 236a, and a bushing 238 fixed about the proximal end portion 236a, The bushing 238 may be a plastic bushing that is adhered to the proximal end portion 236a of the alignment pin 236 via frictional engagement, an adhesive, or any other suitable fastening engagement material or technique that resists movement of the bushing 238 longitudinally along a longitudinal axis of the alignment pin 236.

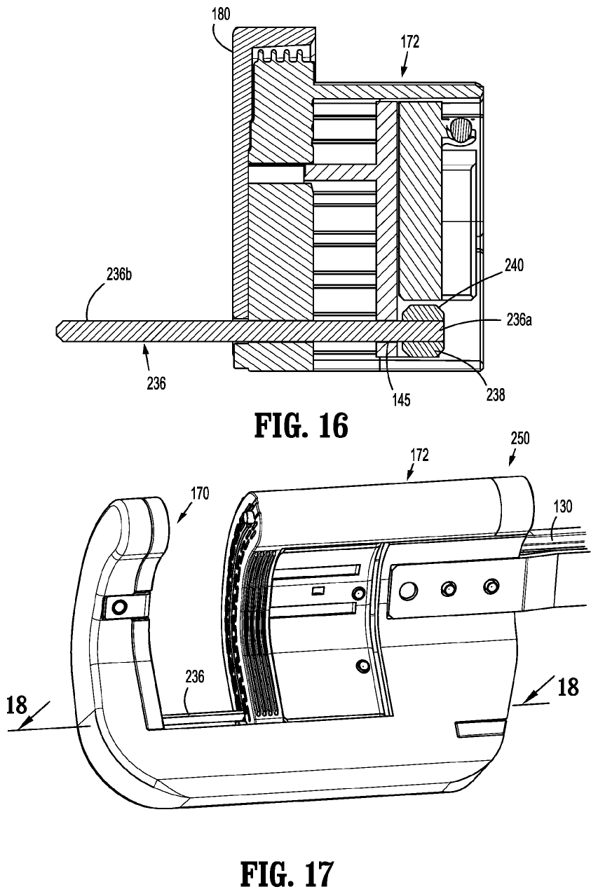

The connection between the bushing 238 and the proximal end portion 236a of the alignment pin 236 is such that a distally-oriented, threshold force exerted on the bushing 238 is designed to overcome the connection to allow for slidable movement of the bushing 238 relative to the alignment pin 236. The bushing 238 has a proximally-facing camming surface 240 configured to engage a distal engagement surface 143 of the head portion 138 of the thrust bar 130. The head portion 138 of the thrust bar 130 defines a longitudinally-extending channel 145 (FIG. 16) configured for receipt of the proximal end portion 236a of the alignment pin 236.

In assembly, with initial reference to FIG. 14, the staple cartridge 172 and the attached shipping cap 180 are inserted into the gap 157 of the housing 152 and the cavity 134 defined between the head portions 132 of the clamp slide members 126a, 126b. While moving the staple cartridge 172 into the housing 152, the chamfered surface 240 of the bushing 238 of the alignment pin 236 engages the head portion 138 of the thrust bar 130 to guide the staple cartridge 172 into the housing 152. With the staple cartridge 172 loaded into the housing 152, the shipping cap 180 is detached from the staple cartridge 172, in the manner described above, to release the alignment pin 236 from the shipping cap 180.

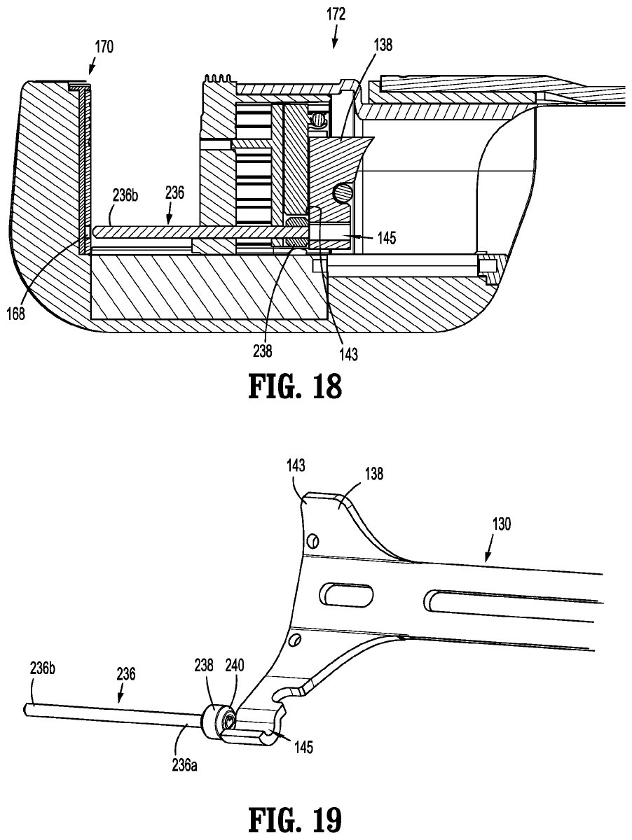

The trigger member 114 (FIG. 1) may be depressed to advance the staple cartridge 172 toward the anvil assembly 170 via the thrust bar 130. As the thrust bar 130 moves distally from a proximal position (FIG. 18) to a distal position, the thrust bar 130 distally translates the bushing 240 along with the alignment pin 236 to position the distal end portion 236b of the alignment pin 236 in the opening 168 (FIG. 18) of the anvil assembly 170.

With the distal end portion 236b of the alignment pin 236 engaged with the anvil assembly 170, and therefore stopped from further distal movement, further advancement of the thrust bar 130 exerts a threshold force on the bushing 238 to release the bushing 238 from the alignment pin 236, thereby permitting further advancement of the thrust bar 130 relative to the alignment pin 236. As the thrust bar 130 advances further (e.g., to carry out a stapling function of the end effector assembly 250), the bushing 238 slides along the alignment pin 236 while the proximal end portion 236a of the alignment pin 236 is passed into the channel 145 of the advancing thrust bar 130.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A curved cartridge assembly, comprising:
a staple cartridge having a tissue-contacting surface, the staple cartridge defining a plurality of rows of staple slots and a channel extending through the tissue-contacting surface;
an alignment pin received in the channel and having a distal end portion that protrudes distally from the tissue-contacting surface of the staple cartridge, the alignment pin movable within the channel from a retracted position to an advanced position; and
a shipping cap detachably coupled to the staple cartridge to cover the tissue-contacting surface of the staple cartridge, the shipping cap having an end portion defining a notch that receives the distal end portion of the alignment pin, wherein the shipping cap engages the alignment pin to resist translation of the alignment pin along a longitudinal axis from the retracted position towards the advanced position.

2. The curved cartridge assembly according to claim 1, wherein the distal end portion of the alignment pin defines a groove, and the end portion of the shipping cap has a tab extending into the notch, the tab received within the groove of the alignment pin to resist translation of the alignment pin from the retracted position towards the advanced position.

3. The curved cartridge assembly according to claim 2, wherein the groove of the alignment pin extends at least partially around a circumference of the alignment pin.

4. The curved cartridge assembly according to claim 1, wherein the staple cartridge further includes a leg extending proximally from an end portion thereof, and the shipping cap further includes:
a plate configured to cover the tissue-contacting surface; and
a leg extending proximally from the plate and configured to detachably secure to the leg of the staple cartridge.

5. The curved cartridge assembly according to claim 1, wherein the shipping cap is configured to detach from the staple cartridge in a direction from a first end portion of the staple cartridge toward a second end portion of the staple cartridge.

6. The curved cartridge assembly according to claim 5, wherein the shipping cap is configured to slide along the tissue-contacting surface of the staple cartridge as the shipping cap is detached from the staple cartridge to disengage from the distal end portion of the alignment pin.

7. An end effector assembly of a surgical stapler, comprising:
a curved housing having a base portion and a jaw portion;
an anvil assembly supported on the jaw portion and having a tissue-contacting surface defining a plurality of staple-forming pockets and an opening;
a curved staple cartridge configured for insertion into the base portion and including a tissue-contacting surface defining a plurality of staple slots;
an alignment pin supported in the curved staple cartridge and having a distal end portion that protrudes distally from the tissue-contacting surface of the curved staple cartridge and is configured for receipt in the opening of the anvil assembly; and
a shipping cap detachably coupled to the staple cartridge and engaged with the distal end portion of the alignment pin to resist axial movement of the alignment pin relative to the curved staple cartridge.

8. The end effector assembly according to claim 7, wherein the distal end portion of the alignment pin defines a groove, and the shipping cap has a tab removably received in the groove.

9. The end effector assembly according to claim 7, wherein the shipping cap is configured to slide along a plane defined by the tissue-contacting surface of the staple cartridge to disengage from the distal end portion of the alignment pin.

10. The end effector assembly according to claim 7, further comprising a slider slidably received in the curved housing, wherein the slider has a camming surface configured to engage a proximal end portion of the alignment pin to resiliently bias the distal end portion of the alignment pin toward the opening in the anvil assembly.

11. The end effector assembly according to claim 10, wherein the proximal end portion of the alignment pin is configured to cam the slider proximally from a distal position to a proximal position during insertion of the staple cartridge into the curved housing.

12. The end effector assembly according to claim 11, wherein the alignment pin is configured to translate distally to position the distal end portion of the alignment pin in the opening of the anvil assembly in response to the slider moving distally from the proximal position to the distal position.

13. The end effector assembly according to claim 12, wherein the slider is configured to move from the proximal position to the distal position in response to the shipping cap being detached from the curved staple cartridge.

14. The end effector assembly according to claim 10, further comprising:
a shaft disposed in the curved housing, the slider slidably supported on the shaft; and
a spring supported on the shaft and engaged with the slider to resiliently bias the slider into engagement with the alignment pin.

15. The end effector assembly according to claim 8, further comprising:
a bushing fixed about a proximal end portion of the alignment pin; and
a thrust bar disposed in the curved housing and configured to move between a proximal position and a distal position, the thrust bar having a distal end portion configured to engage the bushing.

16. The end effector assembly according to claim 15, wherein the bushing and the alignment pin are configured to translate distally to position the distal end portion of the alignment pin in the opening of the anvil assembly in response to the thrust bar moving from the proximal position to the distal position.

17. The end effector assembly according to claim 16, wherein the bushing has a chamfered surface configured to engage the thrust bar during insertion of the curved staple cartridge into the curved housing.

18. The end effector assembly according to claim 16, wherein the thrust bar is configured to unfix the bushing from the alignment pin to move the bushing distally along the alignment pin upon further distal movement of the thrust bar from the distal position.

19. The end effector assembly according to claim 18, wherein the distal end portion of the thrust bar defines a channel configured for receipt of the proximal end portion of the alignment pin as the thrust bar moves distally from the distal position.

20. The end effector assembly according to claim 19, wherein the bushing and the alignment pin move distally as the thrust bar moves distally from the proximal position to the distal position, and the bushing and the thrust bar move distally relative to the alignment pin as the thrust bar moves distally from the distal position.
